(19) United States
(12) Patent Application Publication
(10) Pub. No.: US 2017/0215843 A1
(43) Pub. Date: Aug. 3, 2017

(54) SYSTEM AND METHOD OF PALM-WEARABLE DIAGNOSTIC ULTRASOUND PROBING

(71) Applicant: Whale Imaging, Inc., Waltham, MA (US)

(72) Inventors: Xingbai He, Belmont, MA (US); Changguo Ji, Lexington, MA (US); Xun Zhu, Toronto (CA); Jeff Normand, Worcester, MA (US)

(21) Appl. No.: 15/353,366

(22) Filed: Nov. 16, 2016

Related U.S. Application Data
(60) Provisional application No. 62/388,620, filed on Feb. 3, 2016.

(51) Int. Cl. A61B 8/00; A61B 17/34; A61B 10/02

(52) U.S. Cl. CPC A61B 8/4281; A61B 8/4455; A61B 10/02; A61B 17/3403; A61B 8/4227; A61B 2017/3413

(57) ABSTRACT

An apparatus and corresponding method of use relate to diagnostic ultrasound probing. In particular, the apparatus is suitable for ultrasound guidance for musculoskeletal (MSK) injections. The apparatus is a palm-wearable device configured to removably and replaceably receive and hold a sensor device. The palm-wearable device enables a user to have one hand for manipulating the sensor device and a free hand for performing other tasks such as a MSK injection.

### CROSS-REFERENCE TO RELATED APPLICATION(S)

This application claims priority to, and the benefit of, co-pending U.S. Provisional Application No. 62/388,620, filed Feb. 3, 2016, for all subject matter common to both applications. The disclosure of said provisional application is hereby incorporated by reference in its entirety.

### FIELD OF THE INVENTION

The present invention relates to a medical instrument suitable for diagnostic ultrasound probing. In particular, the present invention relates to a medical instrument suitable for ultrasound guidance for musculoskeletal (MSK) injections.

### BACKGROUND

Generally, ultrasound guided biopsy, injection and surgery, such as musculoskeletal (MSK) injections have been performed based on anatomical landmarks. That is, for performing injections based on anatomical landmarks, the physician feels for specific bones, muscles, or tendons and then injects into the desired area. This technique is known as a “blind” technique because there is no way for the physician to definitively know where the tip of the needle is or where the injection is going. Many providers have been using the landmark or blind technique for a long time and are confident with their skill level. However, MSK ultrasound has become extremely popular, not just for diagnostic imaging of musculoskeletal structures, but the application of ultrasound in pain medicine. Ultrasound is a rapidly growing area for pain management. Ultrasound imaging, uses high frequency sound waves, and provides direct visualization of various soft tissues of the body. As a result of the improved resolution of ultrasound images in the last five years, nerves and nerve tissue is well visualized and bone edges are more identifiable, such that joint spaces are relatively distinguishable.

When combining needle injections with an ultrasound, the needle is easily recognizable and the ultrasound imaging provides real-time needle advancement to the target area. Accordingly, the needle, and needle tip, can be seen on the ultrasound screen and the precise location of the injection is assured (in contrast to the landmark technique). Having an exact location of injection is not only important for the effectiveness of the injection itself, but also for the benefit of the patient and for the feedback to the physician. If the physician knows that the injection has been placed appropriately and the injection does not improve the symptoms, then the physician knows that there must be another cause of the pain described by the patient. Without such a capability, the physician is left guessing as to whether the continued symptoms are due to an inaccurately placed injection, or due to other causes.

In addition to providing the physician with more accurate injection capabilities and improving patient care, currently, there is reimbursement for using ultrasound to visualize the injection site, which motivates use of ultrasound. Ultrasound imaging also better enables patients to understand what is occurring with the injection. Having the patient understand and see the location allows them to become more involved with their own therapy, giving the physician better final results. In addition, the physician can take pre-injection and post-injection images to monitor the progress of the injection treatment.

Another benefit of using ultrasound imaging is that it does not expose the patient and healthcare provider to radiation risks. As an imaging modality, the ability of the ultrasound system to generate a real-time image and real-time loops, or movies, is a benefit to diagnoses. The procedure may be documented, but also the actual motion that the patient goes through to demonstrate their pain or area of pain may be documented. Impingement, for example, can be visualized as it occurs on an ultrasound loop.

The improved performance of ultrasound-guided injections leads to a decrease in health care costs per patient, while improving patient care. Several documented studies have examined the accuracy of this technique. The studies conducted compared the efficacy of blindly guided (landmark technique) versus ultrasound-guided injections. Those studies provide evidence that ultrasound guided injections yield better patient results. The results have shown that ultrasound-guided injections are associated with significantly less procedural injection pain, and increased pain relief lasting for a longer duration than with landmark guided injections. Together these studies have shown that ultrasound-guided injections are associated with significantly less procedural injection pain and provide more pain relief. As individuals embrace wellness and increase their activity level with strenuous movements, repetitive motion, and wear and tear, the result may be painful joints and other musculoskeletal injuries. Physicians will be looking for ways to assist in enabling the healthy lifestyle while decreasing a patient's pain. Beyond physical therapy and other initial techniques to help patients, therapeutic injections are becoming a necessary part of pain management in musculoskeletal injuries. Incorporating diagnostic ultrasound imaging is a tool that improves physician confidence with this form of treatment resulting in better patient care.

A portable ultrasound machine is a medical imaging device that uses ultrasound for diagnostic purposes and is smaller and lighter than the console style ultrasound machines that preceded them. In most cases, such mobile ultrasound systems could be carried by hand and in some cases even operated for a time on battery power alone. Portable ultrasound machines are typically used in situations where space is limited, mobility is important, or the scanning must be done in the field. Currently portable ultrasound machines are used in Cardiac, Vascular, Radiology, Endocrinology, Pediatric, OB/GYN applications and MSK injections.

However, this (technology, device, system, methodology, etc.) experiences some shortcomings. Ultrasound machines are costly and are significant investments for a practice. Similarly, ultrasound machines are not available in large quantities at most practices, so machines may be prioritized for certain procedures causing difficulty scheduling use of the ultrasound machine for procedures in which an alternative treatment is available (e.g., the landmark technique). Additionally, for physicians new to ultrasound, however, there is a learning curve to being able to utilize the ultrasound effectively. Even when familiar with the ultrasound technique, present ultrasound machines still present some challenges for physicians when performing MSK ultrasound. For example, it may be difficult for physicians to use diagnostic ultrasound probing on one hand because some exiting portable ultrasounds are too large to hold and cannot be fixed on the hand properly.

### SUMMARY

There is a need for a palm-wearable diagnostic ultrasound probe for ultrasound-guided injections to solve the technical problems described above. Specifically, the present invention is directed toward solutions to address this need, in addition to having other desirable characteristics. In particular, the present invention provides a palm-wearable diagnostic ultrasound probing device. The device is configured to fit the palm of a physician, and can be used during ultrasound-guided biopsy, injection, and surgeries. The present invention assists medical personnel with conveniently and comfortably manipulating a needle for injection while probing and demobilizing the patient to achieve an accurate and confident injection. The device of the present invention can be applied to biopsy, injection, spinal and a plurality of different surgical procedures.

In accordance with example embodiments of the present invention, a palm-wearable probe device is provided. The device includes a removably and replaceably attached sensor device, a fixation and mounting mechanism holding the sensor device, and a communication interface connected to the sensor device and configured to provide communication from the sensor device to a remote imaging device.

In accordance with aspects of the present invention, the sensor device is one of a linear, a convex, a two-dimensional matrix array, or a matrix transducer array. The sensor device can be an ultrasound sensor device.

In accordance with aspects of the present invention, the fixation and mounting mechanism further includes a strap to hold the palm-wearable probe device in place on a palm of a user.

In accordance with aspects of the present invention, communication interface is one of a probe cable or wireless connection.

In accordance with aspects of the present invention, imaging device is an ultrasound machine.

In accordance with aspects of the present invention, the device further includes an injection point.

In accordance with example embodiments of the present invention, a method for using a palm-wearable probe device is provided. The method includes inserting a sensor device into a fixation and mounting mechanism to form the palm-wearable probe device. The method also includes plugging a communication interface into the sensor device and securing the palm-wearable probe device on a hand of a user, with the sensor device configured at a palm of the user. The method further includes operating the palm-wearable probe device.

In accordance with aspects of the present invention, the operation of the palm-wearable probe device is utilized for at least one of a biopsy, an injection and a surgery.

In accordance with aspects of the present invention, the inserting includes depressing a button on a side of the fixation and mounting mechanism. The inserting can also include releasing the button on the side of the fixation and mounting mechanism to secure the inserted sensor device to the fixation and mounting mechanism.

In accordance with aspects of the present invention, the securing can include wrapping an adjustable strap around a back side of a hand of the user.

In accordance with aspects of the present invention, method further includes removing the sensor device from the fixation and mounting mechanism.

### BRIEF DESCRIPTION OF THE FIGURES

These and other characteristics of the present invention will be more fully understood by reference to the following detailed description in conjunction with the attached drawings, in which:

FIG. 1 is an illustrative depiction of a palm-wearable diagnostic device, in accordance with an embodiment of the present invention;

FIG. 2 is an illustrative depiction of the palm-wearable diagnostic device; and

FIG. 3 is an illustrative flowchart depicting operation of the palm-wearable diagnostic device.

### DETAILED DESCRIPTION

An illustrative embodiment of the present invention relates to a palm-wearable device configured to provide a user with a monitoring and/or probing device during medical procedures while simultaneously keeping a free hand. The palm-wearable probing device includes a sensor device that is removably attached to a mounting mechanism worn on the hand of the user. The mounting mechanism is configured to securely hold the sensor device at a palm location of the user, while simultaneously being secured to the hand of the user.

The palm-wearable device enables the user to have access to a sensor, which can be easily moved and manipulated during various procedures. In an exemplary embodiment, the palm-wearable device can be utilized for ultrasound-guided biopsy, injection and surgeries using an ultrasound sensor in the palm-wearable device. With the ultrasound sensor secured to the palm of the one hand, the user is free to use the other hand to perform the injections or biopsies. In addition to using the palm-wearable device for monitoring or probing, the user also has free use of their fingers on the hand secured with the palm-wearable device. The use of the fingers provides additional benefits, as would be appreciated by one skilled in the art. For example, the user can use the hand with the palm-wearable device to demobilize a patient to achieve an accurate and confident injection with the other hand (e.g., during MSK injections).

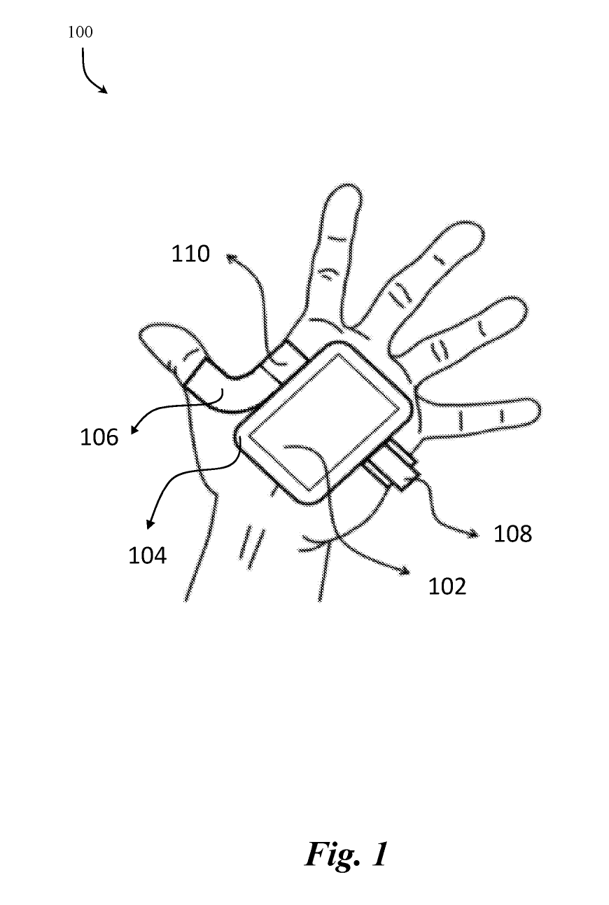

FIGS. 1 through 3, wherein like parts are designated by like reference numerals throughout, illustrate an example embodiment or embodiments of a palm-wearable probe device, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIG. 1 depicts a of palm-wearable probing device 100. The device 100 includes a sensor device 102, a fixation and mounting mechanism 104, a palm ring 106, and communication interface 108. In accordance with an example embodiment of the present invention, the sensor device 102 is an ultrasound sensor that includes linear, convex and/or matrix transducer arrays configured for transmitting ultrasound waves and converting the ultrasound waves into electrical signals. As would be appreciated by one skilled in the art, the sensor device 102 can include any combination of sensors and/or probes known in the art. For example, the sensor device 102 can be any known ultrasound sensor operating as an ultrasound transducer.

In accordance with an example embodiment of the present invention, the sensor device 102 is removably attached to the fixation and mounting mechanism 104. The fixation and mounting mechanism 104 is configured to hold the sensor device 102 in place through a friction fitting. In particular, the fixation and mounting mechanism 104 includes retractable teeth or arms that lock onto of the sensor device 102. The fixation and mounting mechanism 104 also includes a releasing mechanism at an edge of the fixation and mounting mechanism 104, which when depressed retracts the teeth or arms and releases the sensor device 102. As would be appreciated by one skilled in the art, the fixation and mounting mechanism 104 can be configured to be removably attached to a variety of different types of sensor or probe devices and manufactures of those devices. The description of the present invention as it relates to the sensor device 102 are for exemplary purposes and is not intended to limit the present invention to use with a particular device (e.g., an ultrasound sensor). For example, the fixation and mounting mechanism 104 can be universally compatible for use with different ultrasound transducers from different manufacturers.

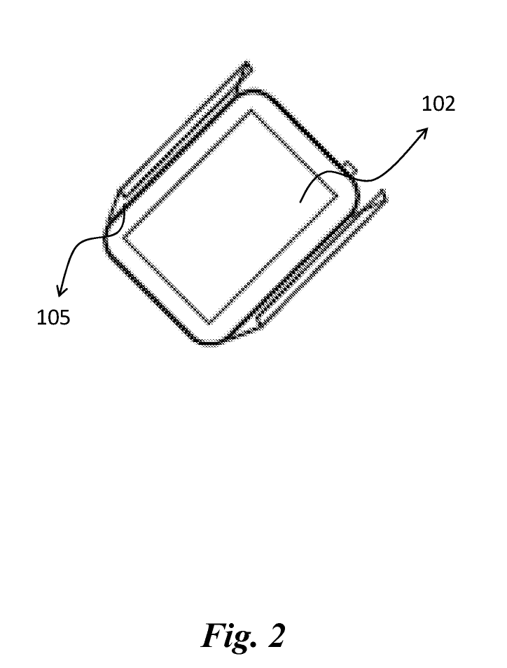

FIG. 2 depicts a view of the device 100 with the teeth or arms 105 of the fixation and mounting mechanism 104 in a releasing position. In this position, the sensor device 102 can be inserted or removed from the fixation and mounting mechanism. The combination of the sensor device 102 and the fixation and mounting mechanism 104 for the device 100. As would be appreciated by one skilled in the art, the sensor device 102 and the fixation and mounting mechanism 104 can be constructed from any combination of flexible and medically preferred materials. For example, the sensor device 102 and the fixation and mounting mechanism 104 can be constructed from a plastic material.

In accordance with an example embodiment of the present invention, the palm ring 106 is shaped and configured to be formed and contoured to fit to an area between a palm and thumb of a user (e,g., a patient), as depicted in FIG. 1. In particular, the palm ring 106 is a curved shaped piece formed to fit around the curve between a thumb and index finger at the palm of the user. Additionally, the palm ring 106 curved shaped piece is curved to form around a palm and a back portion of a hand of the user. The palm ring 106 also includes or is otherwise attached to an adjustable strap 110. The adjustable strap 110 is configured to removably attach the device 100 to a hand of a user and is adjustable to fit different palm sizes of different users. The adjustable strap 110 is connected at each side of the fixation and mounting mechanism 104 and extends around a back side of the hand of the user to hold the device 100 in place. As would be appreciated by one skilled in the art, the palm ring 106 and the adjustable strap 110 can be constructed from any combination of flexible and medically preferred materials. For example, the palm ring 106 and the adjustable strap 110 can be constructed from a flexible nylon material.

In accordance with an example embodiment of the present invention, the communication interface 108 is configured to provide a communication means between the sensor device 102 and an associated machine or monitoring device (not depicted). For example, the communication interface 108 can provide communication means between an ultrasound sensor and an ultrasound machine. In particular, the communication interface 108 includes a probe cable and/or wireless connection to a machine or monitoring device. As would be appreciated by one skilled in the art, the communication interface 108 can include any combination of communication means configured to transfer data from any type of sensor device 102 attached to the fixation and mounting mechanism 104 to a corresponding display, measuring, and/or reading device (e.g., ultrasound machine). The communication interface 108 can include any combination of cables or wireless based communication means. Additionally, the communication interface 108 is configured to extend along a direction of cross section of a palm of the user in a lateral direction to avoid interference between the device 100 and an examining user. The communication interface 108 connects with the sensor device 102.

The device 100 provides a palm-wearable diagnostic sensor device 102 (e.g., an ultrasound sensor) that is held in place by the fixation and mounting mechanism 104. The combination of the palm ring 106 and the adjustable strap 110 hold the combination of the sensor device 102 and the fixation and mounting mechanism 104 in place on a hand of a user. To insert the sensor device 102 into the fixation and mounting mechanism 104, the user depresses a button at an edge of the fixation and mounting mechanism 104 to expend the teeth or arms 105 and insert or release the sensor device 102. The removable aspect of the sensor device 102 from the fixation and mounting mechanism 104 enables a user to exchange one sensor or sensor type with another with minimal effort.

In operation, wearing the device 100 provides users with the convenience to do an examination with the sensor device 102 while keeping a free hand for other usages (e.g., probing, injections, etc.). For example, the device 100 can be worn to provide ultrasound images for use during biopsies, injections, and various surgeries (e.g., MSK injections). With the sensor device 102 fixed on the fixation and mounting mechanism 104 firmly, the user does not have to worry about grabbing or holding a sensor device 102 with their hand and concentrate on performing an examination.

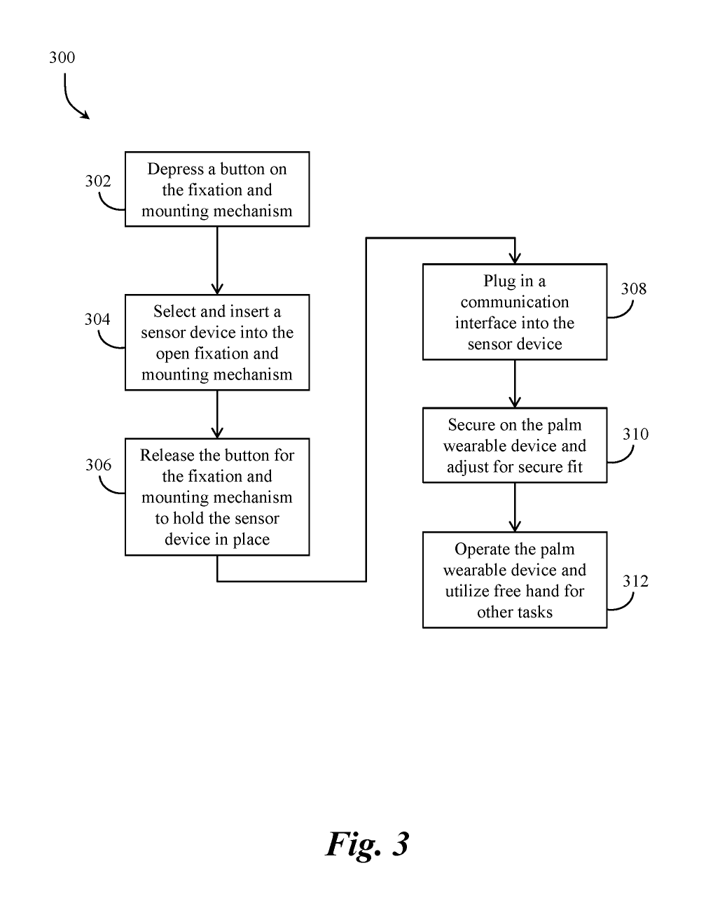

FIG. 3 shows an exemplary flow chart depicting implementation of the present invention. Specifically, FIG. 3 depicts an exemplary flow chart showing the operation of a wearable probing device 100, as discussed with respect to FIGS. 1 and 2. In particular, FIG. 3 depicts a process 300 of configuring and utilizing wearable probing device 100 to examine a patient. At step 302, a user depresses a button on a side of the fixation and mounting mechanism 104 to open the teeth or arms 105 of the fixation and mounting mechanism 104.

At step 304, the user selects and inserts a sensor device 102 into the open fixation and mounting mechanism 104. In an open position the user can slide or clip in the sensor device 102 to be removably attached to the fixation and mounting mechanism 104. At step 306, the user releases the button on the fixation and mounting mechanism 104. Upon releasing the button, the teeth or arms of the fixation and mounting mechanism 104 close and the sensor device 102 is held in place by the teeth or arms of the fixation and mounting mechanism 104.

At step 308, the user plugs a communication interface 108 into a side of the combined sensor device 102 and fixation and mounting mechanism 104. The other end of the communication interface 108 is plugged into a corresponding machine or monitoring device (e.g., ultrasound machine for an ultrasound sensor). The communication interface 108 will provide signals from the sensor device 102 to the corresponding machine or monitoring device during operation.

At step 310, the user secures the combined device 100 (e.g., the sensor device 102 and the fixation and mounting mechanism 104) to their hand. In particular, the user places the combined sensor device 102 and fixation and mounting mechanism 104 in the palm of their hand with the adjustable strap 110 wrapped around the back of their hand. Additionally, the user will place the palm ring 106 of the fixation and mounting mechanism 104 to fit on the curve between their thumb and index finger. With the device 100 on the hand of the user, the user can adjust the adjustable strap 110 to ensure a secure fit of the sensor device 102 on the palm.

At step 312, the user operates the sensor device and optionally performs other tasks with their free hand. In particular, the user can utilize the device 100 to act as a sensor or probe to see beneath the skin of the patient on a nearby monitor (e.g., ultrasound) and use their free hand as they wish. In accordance with an example embodiment of the present invention, the free hand is used for administering injections while viewing the needle on an ultrasound monitor. Overall, the free hand frees the user to perform injections, biopsies, surgeries, etc., with the aid of a sensor device 102 on the other hand.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A palm-wearable probe device, comprising:
a removably and replaceably attached sensor device;
a fixation and mounting mechanism holding the sensor device; and
a communication interface connected to the sensor device and configured to provide communication from the sensor device to a remote imaging device.

2. The palm-wearable probe device of claim 1, wherein the sensor device is one of a linear, a convex, a two-dimensional matrix array, or a matrix transducer array.

3. The palm-wearable probe device of claim 1, wherein the sensor device is an ultrasound sensor device.

4. The palm-wearable probe device of claim 1, wherein the fixation and mounting mechanism further comprises a strap to hold the palm-wearable probe device in place on a palm of a user.

5. The palm-wearable probe device of claim 1, wherein the communication interface is one of a probe cable or wireless connection.

6. The palm-wearable probe device of claim 1, wherein the imaging device is an ultrasound machine.

7. The palm-wearable probe device of claim 1, further comprising an injection point.

8. A method for using a palm-wearable probe device, the method comprising:
inserting a sensor device into a fixation and mounting mechanism to form the palm-wearable probe device;
plugging a communication interface into the sensor device;
securing the palm-wearable probe device on a hand of a user, with the sensor device configured at a palm of the user; and
operating the palm-wearable probe device.

9. The method of claim 8, wherein the operation of the palm-wearable probe device is utilized for at least one of a biopsy, an injection and a surgery.

10. The method of claim 8, wherein the inserting further comprises depressing a button on a side of the fixation and mounting mechanism.

11. The method of claim 10, wherein the inserting further comprises releasing the button on the side of the fixation and mounting mechanism to secure the inserted sensor device to the fixation and mounting mechanism.

12. The method of claim 8, wherein the securing further comprises wrapping an adjustable strap around a back side of a hand of the user.

13. The method of claim 8, further comprising removing the sensor device from the fixation and mounting mechanism.
